(19) United States
(12) Patent Application Publication
(10) Pub. No.: US 2026/0315647 A1
(43) Pub. Date: Oct. 15, 2026

(54) ALKYLENE OXIDE COPOLYMER (AOC) AND THROMBIN FUNCTIONALIZED BONE HEMOSTASIS MATERIAL

(71) Applicants: BAXTER INTERNATIONAL INC., Deerfield, IL (US); BAXTER HEALTHCARE SA, Glattpark (Opfikon) (CH)

(72) Inventors: Justin Robert Daller, Cary, IL (US); Tim Fulghum, Lakemoor, IL (US)

(21) Appl. No.: 19/461,178

(22) Filed: Jan. 27, 2026

Related U.S. Application Data
(60) Provisional application No. 63/750,825, filed on Jan. 29, 2025.

(51) Int. Cl. A61L 24/10; A61L 24/00; A61L 24/04

(52) U.S. Cl. CPC A61L 24/108; A61L 24/0015; A61L 24/0084; A61L 24/043; A61L 2400/04

(57) ABSTRACT

The present disclosure sets forth biocompatible composition for use as a bone hemostat. The composition comprises thrombin and an alkylene oxide copolymer (“AOC”) blend. The thrombin may be sourced from human thrombin, bovine thrombin, or recombinant origin. The AOC blend consists of Poloxamer 188 and Pluriol V-10. The Poloxamer 188 constitutes 45% by weight of the total AOC blend, and the Pluriol V-10 constitutes 55% by weight of the total AOC blend.

### CROSS-REFERENCE TO RELATED APPLICATIONS

The present application claims the benefit of U.S. Provisional Application No. 63/750,825, entitled ALKYLENE OXIDE COPOLYMER (AOC) AND THROMBIN FUNCTIONALIZED BONE HEMOSTASIS MATERIAL and filed Jan. 29, 2025, the contents of which are hereby incorporated by reference in their entirety.

### TECHNICAL FIELD

The present disclosure relates generally to biocompatible materials, and more particularly to compositions for use in biomedical applications and methods of their manufacture.

### BACKGROUND

Bones are living vascular organs which form part of the body's skeleton. Bones may include a variety of tissue types, including marrow, endosteum, periosteum, blood vessels, epithelium, nerves, cartilage, and mineralized osseous tissue. Bleeding from cut or disrupted bone is a common occurrence in many operative procedures. Excessive bleeding from bone during surgery may impair the surgeon's view of the operative field, may result in the need for blood transfusions, and may be associated with post-operative complications.

Cauterization techniques are used to control bleeding in soft tissue but are ineffective for controlling bleeding in bone. Hence, bleeding in bone has traditionally been treated using organic bone wax, a beeswax-based product that can be smeared across the cut surface to plug the holes in the bone, to reduce or stop bleeding. More recently, synthetic bone hemostasis materials have been proposed, including Ostene (Baxter International, Inc.) and Montage (Abyrx, Inc.).

Although bone hemostasis materials are currently available and provide real benefits to patients in need thereof, many advances may still be made to provide improved compositions for bone hemostasis. For example, conventional bone wax materials require improvement due to several limitations. Firstly, they are typically composed of beeswax or synthetic equivalents, which primarily provide mechanical blockage to bleeding but do not actively promote coagulation or facilitate wound healing. This limitation is especially evident in cases where quick or effective hemostasis is needed during surgical procedures. Additionally, these materials may remain in the body indefinitely as they are not resorbable, which can lead to adverse tissue reactions, inflammation, or infection. The non-resorbable nature can also impede new bone growth, hindering the healing process. Moreover, the hydrophobic nature of conventional bone wax may prevent it from adhering effectively to wet bone surfaces, reducing its efficacy under surgical conditions involving bleeding. Finally, the mechanical blockage provided may not be enough in cases of high vascularity or bleeding, necessitating additional methods or materials to achieve hemostasis.

Thus, it is desirable for bone hemostasis materials to provide both sealant properties to physically block blood leakage and coagulation properties to chemically promote blood clotting and halt bleeding. As such, devices and compositions for bone hemostasis with the addition of biological hemostatic agents, such as thrombin, are accordingly needed.

### SUMMARY

To improve the effectiveness of bone hemostat compositions, a new bone hemostat composition is described herein. Specifically, the present disclosure seeks to provide a biocompatible material for use in biomedical applications, and methods for their use and manufacture.

In light of the disclosure set forth herein, and without limiting the disclosure in any way, in a first aspect of the present disclosure, which may be combined with any other aspect, or portion thereof, a biocompatible composition for use as a bone hemostat is provided, the composition comprising thrombin selected from the group consisting of human thrombin, bovine thrombin, or recombinant thrombin; and an alkylene oxide copolymer (“AOC”) blend consisting of Poloxamer 188 and Pluriol V-10, wherein the Poloxamer 188 constitutes 45% by weight of the total AOC blend, and wherein the Pluriol V-10 constitutes 55% by weight of the total AOC blend.

In a second aspect in combination with any prior aspect, the techniques described herein relate to a biocompatible composition, wherein the thrombin is recombinant human thrombin.

In a third aspect in combination with any prior aspect, the techniques described herein relate to a biocompatible composition, wherein the thrombin is present in the biocompatible composition in a range of from about 2,500 to about 5,000 International Units (IU).

In a fourth aspect in combination with any prior aspect, the techniques described herein relate to a biocompatible composition, wherein the biocompatible composition is provided in a paste.

In a fifth aspect in combination with any prior aspect, the techniques described herein relate to a biocompatible composition, wherein the biocompatible composition includes a resorbable polymer matrix and the biocompatible composition is absorbed in a body or a patient within 30 days.

In a sixth aspect in combination with any prior aspect, the techniques described herein relate to a biocompatible composition, wherein the biocompatible composition includes at least one cross-linked gelatin particle.

In a seventh aspect in combination with any prior aspect, the techniques described herein relate to a biocompatible composition, wherein the biocompatible composition includes at least one ceramic particle selected from the group consisting of hydroxyapatite and siliconated hydroxyapatite.

In an eighth aspect in combination with any prior aspect, the techniques described herein relate to a biocompatible composition, wherein the biocompatible composition is configured to be a carrier for an anti-inflammatory drug.

In a nineth aspect in combination with any prior aspect, the techniques described herein relate to a biocompatible composition, wherein the AOC blend includes polyethylene glycol.

In a tenth aspect in combination with any prior aspect, the techniques described herein relate to a system for achieving hemostasis in bone, the system including: a biocompatible composition including thrombin selected from the group consisting of human thrombin, bovine thrombin, or recombinant human thrombin and an alkylene oxide copolymer (AOC) blend consisting of Poloxamer 188 and Pluriol V-10, wherein Poloxamer 188 constitutes approximately 45% by weight and Pluriol V-10 constitutes approximately 55% by weight of a total AOC blend; and a delivery apparatus configured to aid in delivery of the biocompatible composition to a bone surface of a patient, wherein the delivery apparatus is selected from the group consisting of a paste, a dough and a putty.

In an eleventh aspect in combination with any prior aspect, the techniques described herein relate to a system, wherein the thrombin is recombinant human thrombin.

In a twelfth aspect in combination with any prior aspect, the techniques described herein relate to a system, wherein the biocompatible composition is in a ready-to-use form that requires no additional preparation prior to application.

In a thirteenth aspect in combination with any prior aspect, the techniques described herein relate to a system, wherein the delivery apparatus is a paste.

In a fourteenth aspect in combination with any prior aspect, the techniques described herein relate to a system, wherein the system is configured to apply the biocompatible composition across a surface of a bone of a patient evenly.

In a fifteenth aspect in combination with any prior aspect, the techniques described herein relate to a system, wherein the biocompatible composition includes a medicament selected from the group consisting of an antibiotic and an anti-inflammatory agent.

In a sixteenth aspect in combination with any prior aspect, the techniques described herein relate to a method for achieving hemostasis in bone, the method including: applying a biocompatible composition to a bone site, wherein the composition includes: thrombin selected from the group consisting of human thrombin, bovine thrombin and recombinant human thrombin, and an alkylene oxide copolymer (AOC) blend consisting of Poloxamer 188 and Pluriol V-10, with the Poloxamer 188 constituting approximately 45% by weight and the Pluriol V-10 constituting approximately 55% by weight of a total AOC blend; forming a mechanical barrier at the bone site to control bleeding; and facilitating a formation of a stable blood clot at the bone site through an interaction of the thrombin with bleeding tissue.

In a seventeenth aspect in combination with any prior aspect, the techniques described herein relate to a method, wherein the method further includes warming the biocompatible composition to enhance malleability before application to a bone site.

In an eighteenth aspect in combination with any prior aspect, the techniques described herein relate to a method, wherein applying the biocompatible composition includes using a spatula to spread the composition evenly over a bleeding site.

In a nineteenth aspect in combination with any prior aspect, the techniques described herein relate to a method, wherein forming a mechanical barrier includes applying pressure to ensure adherence of the composition to the bone surface.

In a twentieth aspect in combination with any prior aspect, the techniques described herein relate to a method, wherein the biocompatible composition includes additives to accelerate clot formation.

In light of the disclosure set forth herein, and without limiting the disclosure in any way, in a first aspect of the present disclosure, which may be combined with any other aspect, or portion thereof, a biocompatible composition for use as a bone hemostat is provided, the composition comprising thrombin selected from the group consisting of human thrombin, bovine thrombin, or recombinant thrombin; and an alkylene oxide copolymer (“AOC”) blend consisting of Poloxamer 188 and Pluriol V-10, wherein the Poloxamer 188 constitutes 45% by weight of the total AOC blend, and wherein the Pluriol V-10 constitutes 55% by weight of the total AOC blend.

It is another advantage of the present disclosure to provide a ready-to-use bone hemostasis composition that does not require any time consuming or complication pre-use mixing.

In light of the above aspects and present disclosure set forth herein, it is an advantage of the present disclosure to provide a bone hemostat composition for application to a patient's bone during a surgical procedure to act as a mechanical barrier to prevent blood leakage.

It is another advantage of the present disclosure to provide a bone hemostasis composition with improved hemostatic efficacy through the addition of human thrombin, recombinant human thrombin, or thrombin from bovine sources, for controlling bleeding from bone surfaces by functioning as a mechanical barrier, as well as interacting synergistically with thrombin to facilitate the formation of a stable blood clot at the bleeding site.

It is another advantage of the present disclosure to provide a ready-to-use bone hemostasis composition that does not require pre-use mixing.

Additional features and advantages are described in, and will be apparent from, the following Detailed Description. The features and advantages described herein are not all-inclusive and, in particular, many additional features and advantages will be apparent to one of ordinary skill in the art in view of the description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### DETAILED DESCRIPTION

Embodiments of the present invention encompass biocompatible compositions for use in hemostasis. In some cases, bone hemostat compositions may include polyoxyethylene-polyoxypropylene block copolymers, natural polymers, ceramic particles, ethylene glycol polymers, oxazoline polymers, and various combinations or blends thereof. Optionally, bone hemostat compositions may be formulated as aqueous or nonaqueous compositions.

The term “biocompatible material” used herein encompasses a material that does not threaten, impede, or adversely affect living tissue.

The term “resorbable polymer matrix” used herein encompasses a polymer composition which can be gradually dissolved and eliminated from the body.

The term “copolymer” used herein (also known as a heteropolymer) encompasses a polymer derived from two or more types of monomeric species. This is in contrast to a homopolymer where only one type of monomer is used.

The term “non-random” used herein encompasses an intra-chain distribution of co-monomers having a particular pattern that is segmented. It is a unique structural feature of a block copolymer.

The term “poloxamer” used herein encompasses a nonionic triblock copolymer composed of a central hydrophobic chain of polyoxypropylene (poly(propylene oxide)) flanked by two hydrophilic chains of polyoxyethylene (poly(ethylene oxide)). In some instances, a poloxamer may be referred to as a polyoxyethylene-polyoxypropylene (POE-POP) block copolymer.

The term “thrombin” used herein encompasses human-sourced thrombin, bovine-sourced thrombin, or recombinant thrombin.

The term “biological hemostatic agent” refers to an agent that is derived from or designed to mimic naturally occurring biological processes or components to aid in hemostasis.

The term “bone hemostasis” refers to a process of inhibiting, preventing, or otherwise modulating or controlling bleeding in bone. Relatedly, the terms “bone hemostat” encompasses compositions that can be applied or administered to bone, for the intended purpose of achieving or facilitating bone hemostasis.

Each aspect or embodiment described herein may be combined with any other aspect(s) or embodiment(s) unless clearly indicated to the contrary. In particular, any feature indicated as being preferred or advantageous may be combined with any other feature or features indicated as being preferred or advantageous.

The biocompatible material described herein is suitable for use in therapy. Such therapy includes, but is not limited to, medicine, dentistry, and surgery. More specific applications include use of the polymer composition as a hemostatic agent or a carrier for medication.

In exemplary biocompatible materials, a resorbable polymer matrix can provide a continuous phase, and additives or particles can provide a non-continuous, dispersed phase.

Bone hemostat compositions as disclosed herein can be administered to cut or damaged bone of a patient for controlling, inhibiting, or preventing bleeding from the bone. Exemplary compositions for use in bone hemostasis include synthetic, resorbable, or soluble (e.g., water soluble) polymers that after application to a bleeding bone surface or site will temporarily remain in place to provide the desired effect of controlling blood flow, and will then dissolve, disperse, or otherwise disappear from the treatment area within a few days, thus leaving space for new bone to grow. In some instances, a bone hemostat composition may be provided in a paste or paste-like form. In some instances, a bone hemostat composition may be provided in a doughy or dough-like form. Further, a bone hemostat composition may be provided in a putty or putty-like form. According to some embodiments, a bone hemostat composition may be provided in a nonaqueous form that is a viscous paste and dough-like.

Exemplary bone hemostat compositions may include granules, flakes, powders, or various combinations thereof, or any hemostatic and biodegradable components, and may be provided as molding compounds for bone or bone tissue. Exemplary bone hemostat compositions may optionally include cross-linked gelatin particles, alone or in combination with ceramic particles such as particles of hydroxyapatite or Si-hydroxyapatite, in a moldable, malleable carrier based on polymer compositions or blends.

Various formulations based on non-random and random copolymer compositions, including without limitation Poloxamer 188, also known as Pluronic F68, Poloxamer 407, Pluronics with hydrophobic properties (i.e., L-31, L61), and Pluriol V-10, along with other polymer components such as polyethylene glycol of various molecular weights, can be used in the preparation of polymer compositions or blends for a bone hemostat or bone hemostat with enhanced hemostatic and osteogenic properties.

Bone hemostat compositions with enhanced properties may also include gelatin and cross-linked derivatives thereof, such as, for example, cross-linked gelatin particles, as a filler. In some embodiments, inorganic particles based on nanosize hydroxyapatite, siliconated hydroxyapatite biphasic ceramic, and the like, have been used as an osteogenic filler for bone hemostat.

In addition to, or alternatively, bone hemostat compositions with enhanced properties may include the addition of a biological hemostatic agent, such as, for example, thrombin sourced from human, recombinant, or bovine origin. Thrombin is a pro-coagulant enzyme with a primary role of catalyzing the conversion of fibrinogen to fibrin, which is crucial in forming blood clots. Specifically, thrombin activates coagulation Factor V, Factor VIII, Factor XIII, and platelets, and enzymatically converts fibrinogen into fibrin monomer which rapidly reacts and polymerizes to form fibrin polymer, which forms a clot. The formation of the clot, alone, is beneficial to help seal the wound and stop bleeding more quickly. However, the formation of this clot is also beneficial because it may promote stronger adhesion to the bone upon application of the composition. For example, the blood clot that forms may act as a binding agent for quicker and stronger adhesion of the composition to the bone, which is particularly useful for surgical wounds or lacerations where quick tissue sealing is necessary to promote faster healing and reduce the risk of infection. Moreover, the addition of thrombin to the bone composition is beneficial because fibrin, the byproduct of thrombin's action, is a natural component of the extracellular matrix that is essential for tissue repair. Thus, adding thrombin to the bone composition of the present invention may promote tissue regeneration at the wound site and accelerate the tissue repair process.

Exemplary bone hemostat compositions may be provided as a malleable paste. For example, embodiments encompass a malleable, ready-to-use hemostat for application in bone. Exemplary bone hemostat compositions may include a synthetic polymer matrix, optionally combined with hemostatic agents, antibiotics, or ceramic particles or powders. Bone hemostat paste compositions disclosed herein may be provided in a ready-to-use form for bone hemostasis, that do not require any preparation such as pre-warming or kneading. Exemplary bone hemostat compositions exhibit a reasonably short dissolution time. Exemplary bone hemostat compositions disclosed herein can be formulated so as to not change their handling characteristics upon kneading or application to a bone site, which may be bleeding, damaged, or otherwise compromised.

Embodiments of the present invention encompass bone hemostat compositions that stop or inhibit bone bleeding upon application, resist irrigation, and remain in place for a duration sufficient to achieve stable hemostasis. Exemplary bone hemostat compositions are formulated to offer controlled, precise application, and to conform to the site of care. In some embodiments, bone hemostat compositions may be ready for use directly out of the package, and do not require warming or kneading prior to application to the patient's bone. In some instances, bone hemostat compositions as disclosed herein are resorbed or biodegraded in the patient's body within 30 days. Exemplary bone hemostat compositions can permit normal bone healing and promote bone regeneration. In some instances, bone hemostat compositions can reduce hematoma formation. Moreover, exemplary bone hemostats can be used as carriers for medications including anti-inflammatory drugs which may be used to reduce inflammation at the surgical site. Embodiments of the present invention provide polymeric formulations with superior handling properties for bone hemostasis.

### Primary Non-Random Copolymer Components

Embodiments of the present invention encompass compositions having one or more primary non-random copolymer components. For example, bone hemostat compositions may include at least one non-random copolymer of poly(alkylene oxide)s or derivatives thereof. A non-random copolymer of poly(alkylene oxide)s can be linear or branched. Exemplary poly(alkylene oxide)s may include polyoxyethylene-polyoxypropylene (POE-POP) block copolymers or poloxamers, and may contain poly(ethylene oxide) (EO) and poly(propylene oxide) (PO) units with the molecular formula (EO)ₓ(PO)_{y}(EO)ₓ. A non-random copolymer may include at least two types of alkylene oxides, such as poly(ethylene oxide) (EO) and poly(propylene oxide) (PO), forming the poly(alkylene oxide) blocks in the copolymer. In some embodiments, a non-random copolymer of poly(alkylene oxide)s has a number average molecular weight within a range from about 6,500 to about 16,300 g/mol. In some cases, the molecular weight may be determined based on an end group analysis approach. The number average molecule weight of the poly(alkylene oxide) may be selected so as to confer certain handling properties, such as a desired deformation force or working window, to the bone hemostat composition. A non-random copolymer, for example, in conjunction with other components of the bone hemostat composition may operate to facilitate blood wicking and clotting.

In some embodiments, the bone hemostat composition may include Poloxamer 188 (Pluronic F68), which is a commercially available polyoxyethylene-polyoxypropylene triblock copolymer of the formula (EO)₂₀(PO)₇₀(EO)₂₀. Alternatively, in some embodiments, the bone hemostat composition may include Poloxamer 407 (Pluronic F127), which is a commercially available polyoxyethylene-polyoxypropylene triblock copolymer of the formula (EO)₁₀₁(PO)₅₆(EO)₁₀₁. Depending on the values of the x and y in the formula (EO)ₓ(PO)_{y}(EO)ₓ, the molecular weight and/or the PO percentage of the poloxamer may vary.

### Basic Matrix for Soluble Bone Hemostat

Embodiments of the present invention encompass the use of various polymeric compositions to provide a basic matrix for soluble bone hemostat composition. In some embodiments, one or more primary non-random copolymer components can be used to prepare such a basic matrix. Relatedly, a one or more primary non-random copolymer components, optionally in combination with one or more secondary non-random copolymer components, and/or one or more random copolymer components, can be mixed at various ratios to provide a basic matrix for soluble bone hemostat. For example, it is possible to prepare a basic matrix for soluble bone hemostat by mixing Poloxamer 188 and Pluriol V-10 at selected ratios, as discussed elsewhere herein.

A primary non-random copolymer component (e.g. Pluronic F127) may be a solid polymer that after melting will form a hard and brittle material. A random copolymer component (e.g., Pluriol V-10) may be a very viscous polymer with hydrophobic properties, and when blended with a primary non-random copolymer component (e.g., Poloxamer 188) may provide a resulting material with good handling properties, such as being soft and malleable.

Exemplary compositions of the bone hemostat composition may include an alkylene oxide copolymer (“AOC”) blend of Poloxamer 188 and Pluriol V-10. Further, in a preferred embodiment, the AOC blend comprises 45% Poloxamer 188 by total weight of the composition, and 55% Pluriol V-10 by total weight of the composition.

The presence of hemostatic additives in poloxamer compositions can improve or provide blood wicking and clotting at the local defect site. Poloxamers may operate as a mechanical barrier, by obstructing the bleeding vessels. Polymeric compositions or blends can be used alone, or in some instances as a carrier or a matrix for antibiotics, blood coagulants or related materials including natural polymer components such as gelatin and cross-linked derivatives, for example cross-linked gelatin particles, or osteoconductive ceramics.

In a preferred embodiment, the polymeric blend is used with a thrombin constituent. The thrombin may of human source or bovine source, or the thrombin may be recombinant thrombin. The thrombin may be topically applied to the bone hemostat composition of the present invention and/or the thrombin may be mixed throughout the bone hemostat composition. The bone hemostat composition with thrombin is in a ready-to-use formulation and does not require pre-mixing before use. For example, in a preferred embodiment the bone hemostat composition is an AOC blend of Poloxamer 188 and Pluriol V-10 and RECOTHROM recombinant human thrombin, available from Baxter Healthcare Corporation. The thrombin may be present in the composition in a range of 2,500 to 5,000 International Units (IU). The composition is capable of being warmed to a desired consistency using an aseptic technique, and then may be manipulated and softened with dry, gloved fingers, such as a physician's finger's prior to applying it to a patient's wound during surgery.

According to embodiments of the present invention, one or more primary non-random copolymer components may be used, optionally in combination with other components as described elsewhere herein, to prepare a matrix for a bone hemostat putty. The solubility of non-random copolymer components, optionally in combination with other components as described elsewhere herein, can be tailored to obtain a bone hemostat composition with desired dissolution kinetics, for example by blending polymers with different hydrophilic-hydrophobic properties or by blending soluble and poorly soluble polymers.

1. A biocompatible composition for use as a bone hemostat, the biocompatible composition comprising:
thrombin selected from the group consisting of human thrombin, bovine thrombin, or recombinant human thrombin; and
an alkylene oxide copolymer (“AOC”) blend consisting of Poloxamer 188 and pluriol V-10, wherein the Poloxamer 188 constitutes 45% by weight of a total AOC blend, and wherein the Pluriol V-10 constitutes 55% by weight of the total AOC blend.

2. The biocompatible composition of claim 1, wherein the thrombin is recombinant human thrombin.

3. The biocompatible composition of claim 1, wherein the thrombin is present in the biocompatible composition in a range of from about 2,500 to about 5,000 International Units (IU).

4. The biocompatible composition of claim 1, wherein the biocompatible composition is provided in a paste.

5. The biocompatible composition of claim 1, wherein the biocompatible composition includes a resorbable polymer matrix and the biocompatible composition is absorbed in a body or a patient within 30 days.

6. The biocompatible composition of claim 1, wherein the biocompatible composition comprises at least one cross-linked gelatin particle.

7. The biocompatible composition of claim 1, wherein the biocompatible composition comprises at least one ceramic particle selected from the group consisting of hydroxyapatite and siliconated hydroxyapatite.

8. The biocompatible composition of claim 1, wherein the biocompatible composition is configured to be a carrier for an anti-inflammatory drug.

9. The biocompatible composition of claim 1, wherein the AOC blend includes polyethylene glycol.

10. A system for achieving hemostasis in bone, the system comprising:
a biocompatible composition including thrombin selected from the group consisting of human thrombin, bovine thrombin, or recombinant human thrombin and an alkylene oxide copolymer (AOC) blend consisting of Poloxamer 188 and Pluriol V-10, wherein Poloxamer 188 constitutes approximately 45% by weight and Pluriol V-10 constitutes approximately 55% by weight of a total AOC blend; and
a delivery apparatus configured to aid in delivery of the biocompatible composition to a bone surface of a patient, wherein the delivery apparatus is selected from the group consisting of a paste, a dough and a putty.

11. The system of claim 10, wherein the thrombin is recombinant human thrombin.

12. The system of claim 10, wherein the biocompatible composition is in a ready-to-use form that requires no additional preparation prior to application.

13. The system of claim 10, wherein the delivery apparatus is a paste.

14. The system of claim 10, wherein the system is configured to apply the biocompatible composition across a surface of a bone of a patient evenly.

15. The system of claim 10, wherein the biocompatible composition includes a medicament selected from the group consisting of an antibiotic and an anti-inflammatory agent.

16. A method for achieving hemostasis in bone, the method comprising:
applying a biocompatible composition to a bone site, wherein the composition comprises:
thrombin selected from the group consisting of human thrombin, bovine thrombin and recombinant human thrombin, and an alkylene oxide copolymer (AOC) blend consisting of Poloxamer 188 and Pluriol V-10, with the Poloxamer 188 constituting approximately 45% by weight and the Pluriol V-10 constituting approximately 55% by weight of a total AOC blend;
forming a mechanical barrier at the bone site to control bleeding; and
facilitating a formation of a stable blood clot at the bone site through an interaction of the thrombin with bleeding tissue.

17. The method of claim 16, wherein the method further comprises warming the biocompatible composition to enhance malleability before application to a bone site.

18. The method of claim 16, wherein applying the biocompatible composition includes using a spatula to spread the composition evenly over a bleeding site.

19. The method of claim 16, wherein forming a mechanical barrier includes applying pressure to ensure adherence of the composition to the bone surface.

20. The method of claim 16, wherein the biocompatible composition includes additives to accelerate clot formation.
